(19) United States
(12) Patent Application Publication
(10) Pub. No.: US 2006/0154993 A1
(43) Pub. Date: Jul. 13, 2006

(54) Method for treating and preventing male infertility

(76) Inventors: Gian Paolo Littarru, Ancona (IT); Giancarlo Balercia, Ancona (IT)

Correspondence Address: MCCARTER & ENGLISH LLP;CITYPLACE I, 185 ASYLUM STREET, HARTFORD, CT, 06103, US

(21) Appl. No.: 11/300,996

(22) Filed: Dec. 15, 2005

Related U.S. Application Data
(60) Provisional application No. 60/637,420, filed on Dec. 17, 2004.

(51) Int. Cl. A61K 31/12

(52) U.S. Cl. 514/690

(57) ABSTRACT

The present invention relates to a novel method of treating male infertility such as, for example, idiopathic asthenospermia. In one aspect the present invention relates to the treatment or prevention of male infertility through the administration of a composition containing ubiquinone, ubiquinol or both, and optionally, a member chosen from the group consisting of a reducing agent, a solubilizer, an emulsifier, a surfactant, a triglyceride, an oil, a fat, a lipid, a phospholipid, a carbohydrate, an antioxidant, a carnitine, creatine, an herbal extract, a vitamin, a coenzyme, a mineral, an electrolyte, a pharmaceutical, a water, a carrier, an excipient, and any combination thereof.

### CROSS-REFERENCE TO RELATED APPLICATIONS

Under 35 U.S.C. § 119(e), this application claims the benefit of U.S. Provisional Application No. 60/637,420 filed Dec. 17, 2004, which is hereby incorporated by reference in its entirety.

### FIELD OF INVENTION

The present invention relates to a method of treating or preventing male infertility, for example, idiopathic asthenospermia, through the administration of an effective amount of coenzyme Q10 in a pharmaceutically acceptable form. The invention also relates to the administration of an effective amount of coenzyme Q10 in a pharmaceutically acceptable form to improve the oxidative and bioenergetic capacity of sperm cells, and seminal plasma.

### BACKGROUND

Co-enzyme Q10 (CoQ10 or ubiquinone) is a vitamin-like component of the mitochondrial respiratory chain and plays a crucial role in energy metabolism. Humans synthesize a limited quantity of ubiquinone, additional CoQ10 is consumed as part of the diet. CoQ10 plays a central role in the cells during the production of energy. Furthermore, it functions as an antioxidant in the body's lipid phase in much the same manner as vitamin E and various carotenoids.

CoQ10 possesses membrane-stabilizing properties, and is the best known member of a group of lipophilic quinones that display electron transfer activity within the lipid phase of cellular membranes. CoQ10 is also an important liposoluble chain-breaking antioxidant associated with membranes and lipoproteins. Other quinones of this general lipophilic type that are found in cells include, for example, chlorobiumquinone, menaquinone, phylloquinone, plastoquinone, rhodoquinone, and thermoplasmaquinone. While CoQ10 has commonly been used to treat congestive heart failure and other cardiac problems, recent evidence indicates a role in the male reproductive system.

In particular, an excess of reactive oxygen species (ROS) by abnormal spermatozoa and leukocytes is known to impair sperm cell function and play a negative role in male factor fertility. The consequent impairment of sperm function can be attributed to, for example, concomitant decreases in sperm motility and/or impaired membrane integrity induced by lipid peroxidation, both of which are key contributors to male factor infertility.

In addition, CoQ10 biosynthesis is active in testis and high levels of its reduced form, QH2 (ubiquinol), are present in semen. Furthermore, a negative correlation between ROS excess and incidence of spontaneous pregnancy has been shown. On the other hand, some data support a reduced total scavenging capacity of seminal plasma in infertile men with abnormal semen parameters. The role of CoQ10 as a lipophilic antioxidant has been well established in plasma lipoproteins and a role in male factor infertility is possible. Thus, CoQ10 may act as a free radical scavenger in this biological system.

There is also evidence that sperm cells with reduced motility have a significant reduction in the phospholipid pool, as well as phosphatidylethanolamine (PE) and phosphatidylcholine (PC) content, probably related to a reduction in the antioxidant capacity of spermatozoa and seminal plasma. Moreover, we recently found a significant reduction of CoQ10 levels in the seminal plasma and sperm cells of infertile men with idiopathic and varicocele-associated asthenospermia, which suggests a pathogenetic role through the impairment of total antioxidant reserve.

Some antioxidant molecules (vitamins, gluthatione, carnitine) have been used therapeutically in patients with semen quality impairment and infertility; their real effectiveness remains uncertain at present, mainly because of the indiscriminate selection of patients undergoing treatment, as pointed out by several investigators. Recent data from a double-blind controlled study in selected cases of male factor infertility and an open study of a selected group of infertile men with prostato-vesiculo-epididymitis suggest that a therapeutic approach with carnitine could reduce male factor infertility.

Therefore, there exists a need for a natural dietary supplement that is better tolerated than synthetic pharmaceuticals, and is formulated with natural ingredients including CoQ10, which are beneficial for improving the oxidative state of sperm, and treating male infertility.

### SUMMARY OF THE INVENTION

This application claims the benefit of U.S. Provisional Application No. 60/637,420 filed Dec. 17, 2004, which is hereby incorporated by reference in its entirety.

The present invention relates to a novel method of treating male infertility. In one aspect the present invention relates to the treatment or prevention of male infertility through the administration of a composition containing ubiquinone, ubiquinol or both. In another aspect, the effective amount of coenzyme Q10 (ubiquinone, or ubiquinol) to be administered is from about 40 mg/day to about 4 g/day. In certain aspects of the present invention the effective amount of coenzyme Q10 to be administered is from about 100 mg/day to about 1600 mg/day. In yet other aspects of the present invention the effective amount of coenzyme Q10 to be administered is from about 200 mg/day to about 800 mg/day.

In another aspect, the invention relates to the administration of a composition containing an effective amount of coenzyme Q10 to a male in need thereof for the purpose of scavenging reactive oxygen species (ROS) that negatively affect sperm function. In another aspect, the invention relates to increasing the CoQ10 levels in the seminal plasma, sperm cells or both for the purpose of improving semen parameters, such as concentration, PC content, oxidative capacity, or any combination thereof. A further aspect of this invention relates to improving a sperm cell's health, viability, morphology or teratospermia, kinetic features, treating or preventing idiopathic asthenospermia or any combination thereof. Sperm kinetic features include, for example, forward motility, curvilinear velocity, and straight progressive velocity.

In another aspect of the methods of the current invention, the CoQ10 is administered in a composition also containing a member chosen from the group consisting of a reducing agent, a solubilizer, an emulsifier, a surfactant, a triglyceride, an oil including, for example a vegetable oil, mineral oil or essential oil, a fat, a lipid, a phospholipid, a carbohydrate, an antioxidant, a carnitine, creatine, an herbal extract, a vitamin, a coenzyme, a mineral, an electrolyte, a salt, another biologically active agent, water, a carrier, an excipient, and any combination thereof.

A further aspect of the present invention contemplates a suitable method of delivery of the composition that contains an effective amount of CoQ10 including for example, oral administration in the form of a tablet, capsule, controlled release capsule, gel capsule, paste, cream, nutrient bar, liquid, emulsion or any combination thereof. These examples are given by way of illustration only, and are not to be construed as limiting. Other routes of administration that are within the contemplation of the invention includes, subcutaneous, enteral, parenteral, anal, topical, and the like. Yet another aspect of the invention relates to single or multiple doses of an effective amount of the CoQ10 composition of the present invention in a hermetically sealed packet, a suppository, a pill, a tablet, a capsule including a gelatin capsule, a soft gel capsule, a controlled or extended time release capsule, and any combination thereof.

The present invention provides novel methods for the treatment and prevention of male infertility, such as, for example, idiopathic asthenospermia. The present invention provides a natural alternative for treating or preventing male infertility without the need for synthetic pharmaceuticals that can have potentially serious side effects. Other features, objects and advantages of the invention and its preferred embodiments will become apparent to those of ordinary skill in the art from the detailed description, examples, figures, and claims that follow.

### DETAILED DESCRIPTION OF THE PREFERRED EMBODIMENTS

The present invention provides a new and useful method for treating or preventing male infertility. As used herein, male infertility includes, for example, idiopathic and varicocele-associated asthenospermia, teratospermia, infertility caused by poor sperm kinetics, abnormal sperm morphology, impaired oxidative or bioenergetic capacity of sperm cells or seminal plasma, or any combination thereof.

In a preferred embodiment, the method of the present invention includes administering an effective amount of CoQ10 for the purpose of treating or preventing male infertility. In one embodiment, the effective amount of CoQ10 to be administered is from about 40 mg/day to about 4 g/day. In another embodiment the effective amount of CoQ10 to be administered is from about 100 mg/day to about 1600 mg/day. In a preferred embodiment of the present invention the effective amount of CoQ10 to be administered is from about 200 mg/day to about 800 mg/day.

In another embodiment, the present invention includes administering an effective amount of CoQ10 for the purpose of increasing the level of CoQ10 in the seminal plasma, sperm cells or both of a male in need thereof. The effective dosage of CoQ10 to be administered is from about 40 mg/day to about 4 g/day, more preferably from about 100 mg/day to about 1600 mg/day, and even more preferably from about 200 mg/day to about 800 mg/day.

In another embodiment, the present invention includes administering an effective amount of CoQ10 for the purpose of increasing the levels of PC in the seminal plasma, sperm cells or both of a male in need thereof. In one aspect of this embodiment, the effective dosage of CoQ10 to be administered is from about 40 mg/day to about 4 g/day. In another embodiment, the effective dosage of CoQ10 to be administered is from about 100 mg/day to about 1600 mg/day. In a preferred embodiment of the present invention the effective amount of CoQ10 to be administered is from about 200 mg/day to about 800 mg/day.

In any of the embodiments described above, the dosage of CoQ10 may be administered in any manner that would be familiar to those of ordinary skill in the art, including, for example, oral administration in the form of a tablet, capsule, controlled release capsule, gel capsule, paste, cream, nutrient bar, liquid, emulsion or any combination thereof. These examples are given by way of illustration only, and are not to be construed as limiting. Other routes of administration that are within the contemplation of the inventors includes, subcutaneous, enteral, parenteral, anal, topical, and the like. Yet another aspect of the invention relates to single or multiple doses of an effective amount of the coenzyme Q10 composition of the present invention in a hermetically sealed packet, a suppository, a pill, a tablet, a capsule including a gelatin capsule, a soft gel capsule, a controlled or extended time release capsule, and any combination thereof. The controlled or extended release CoQ10 composition or the method of the invention can be created by any of the techniques known to those of ordinary skill in the art.

In addition, in any of the embodiments of the methods of the invention, the CoQ10 composition may optionally include one or more of the following: a reducing agent, a solubilizer, an emulsifier, a surfactant, a triglyceride, an oil, a fat, a lipid, a phospholipid, a carbohydrate, an antioxidant, lipoic acid, a carnitine, creatine, an herbal extract, a vitamin, a coenzyme, a mineral, an electrolyte, another biologically active agent, for example, a drug or pharmaceutical or the like, water, a carrier, an excipient, and any combination thereof.

Having generally described the invention, reference is now made to the following example which is intended to illustrate preferred embodiments and comparisons. Other features and benefits will be readily apparent to one of ordinary skill in the art, and the following example is not to be construed as limiting the scope of this invention as is more broadly set forth above and in the claims.

### EXAMPLE #1

Heretofore, it was unknown whether the exogenous administration of CoQ10 could lead to any modification of its content in semen or to any benefit on sperm cell function. To elucidate a potential therapeutic role, semen parameters were monitored, including, CoQ10, and PC changes in idiopathic asthenozoospermic infertile patients undergoing CoQ10 dietary supplementation.

Patient Selection

Twenty-two patients (mean age, 31 years; range, 25-39 years) with idiopathic asthenospermia were enrolled in the study. The patients were selected at the Andrology Unit of the Division of Endocrinology, Umberto I Hospital, University of Ancona, Ancona, Italy. All subjects underwent medical screening, including history and clinical examination, and presented with a clinical history of primary infertility of at least 3 years. Testicular volume was evaluated in each patient using Prader's orchidometer. To accomplish a complete diagnosis, the following investigations were also performed: semen analysis; Mar-test (SperMar test, Diasint, Florence, Italy) for anti-spermatozoa antibodies (Ab); sperm culture and urethral specimens collection for Chlamydia and Mycoplasma ureoliticum detection; FSH, LH, T, E2, and PRL assays, using commercial RIA kits; and testicular, prostatic, and seminal vesicle ultrasound and echo-color Doppler of venous spermatic plexus for anatomical abnormalities and varicocele detection.

There was no apparent female factor, since all partners (mean age, 28 years; range, 23-35 years) were ovulating regularly as formally proven by luteal phase progesterone (P) levels and no abnormal fallopian tube anatomy was detected after hysterosalpingography.

Eligibility Criteria

The following criteria were adopted for patient eligibility: (1) sperm count <20×10⁶/mL, sperm motility (forward motility, class a and b, according to World Health Organization (WHO) 1999 criteria), <50% at two distinct sperm analyses, and normal sperm morphology <50%; (2) seminal white blood cells (WBC) <1×10⁶/mL, negative sperm culture, and Chlamydia and Mycoplasma ureoliticum detection; (3) normal serum levels of gonadotropins, T, E2, and PRL; (4) absence of infectious genital diseases, anatomical abnormalities of the genital tract including varicocele, and anti-spermatozoa Ab; (5) absence of systemic diseases or treatment with other drugs in the 3 months before enrollment in the present study; and (6) absence of smoking, alcohol, drug addiction, or occupational chemical exposure.

Study Design And Treatments

The enrolled patients underwent dietary implementation of CoQ10 (PharmaNord, Veyle, Denmark), 200 mg/day twice daily orally for 6 months. Clinical examination, semen analysis including computer-assisted sperm analysis (CASA), and CoQ10 and PC assays were performed at baseline and after 6 months of therapy. An additional semen analysis was performed 6 months after the termination of therapy (wash-out). This study was approved by the Institutional Review Board of the University of Ancona-Umberto I Hospital. All patients provided their written informed consent and completed the entire trial.

Safety Assessment

Safety assessment included medical history, physical examination, hematological screening, and serum chemistry at all visits and the monitoring of drug-related adverse events by recordation in patient diaries.

Seminal Fluid Analysis

Semen quality was assessed by the same biologist in terms of sperm concentration, motility, and morphology in accordance with WHO criteria. Briefly, seminal fluid was obtained by masturbation after 3-5 days of sexual abstinence. The samples were kept in the andrology lab at room temperature and processed within 1 hour from ejaculation. Sperm count was determined with the Makler chamber. Motile spermatozoa were assessed by phase contrast microscopy (10 μL of semen was delivered onto a glass slide and covered with a 22 mm×22 mm coverslip) and graded as follows: class a and b, fast and weak forward motility; class c, nonprogressive motility; class d, immobile spermatozoa. Sperm morphology was evaluated on smears of seminal fluid, stained with the Giemsa method, and observed by oil immersion light microscopy. Conventional immunocytochemistry was used to assess WBC.

In addition, CASA for sperm cell motility was performed, as reported elsewhere. One semen aliquot (3 μL) was placed in a 20-μm depth chamber. Two 20-μm depth cell-VU chambers (Conception Technologies, La Jolla, Calif.) were loaded, and six different fields per chamber were randomly examined and at least 200 sperm for each field of the chamber were scored. Percentages of motile sperm and movement characteristics were analyzed using an automated analyzer at 37° C. (CellTrack VP 110, Motion Analysis Corporation, Palo Alto, Calif.). Sperm velocity and kinetic characteristics were evaluated only for motile sperm and expressed as mean values considering curvilinear velocity (VCL) and straight progressive velocity (VSL).

Preparation For High Pressure Liquid Chromatography Analysis

The liquefied semen samples were centrifuged at room temperature at 500×g for 18 minutes. The sperm cell pellet was washed with 0.15 M NaCl. Two hundred fifty microliters of 1-propanol were added to 50 μL of a cellular suspension containing 10⁶ sperm cells or to 50 μL of seminal plasma. After mixing for 1 minute, the samples were centrifuged for 5 minutes at 2,000×g and the obtained supernatants were injected into the high pressure liquid chromatography (HPLC) apparatus. This extraction assured a total recovery of liposoluble antioxidants.

Analysis of PC was performed according to Frei et al. with the modification described by Yamamoto et al. After adding 1 mL of methanol to a cellular suspension containing about 10⁶ sperm cells or to 50 μL of seminal plasma, samples were mixed for 1 minute; then 6 mL of hexane were added and samples were mixed again before centrifugation (10 minutes at 500 g). Supernatant was collected and dried with a nitrogen stream. Dried sample residues were dissolved in 300 μL of ethanol.

Determination of CoQ10

CoQ10 levels were assayed in sperm cells and seminal plasma using a Beckman Gold HPLC System (Beckman Instruments, San Ramon, Calif.) equipped with an electrochemical detector (EC, ESA 5100, Bedford, Mass.) as described elsewhere. The detector was supplied with a guard cell and an analytical cell with two electrodes in series; the potentials applied to the electrodes were, respectively, +0.6V, −0.45V, and +0.6V; chromatograms were recorded from the second analytical cell signal. One hundred microliters of samples were directly injected into the analytical column and analyzed; an analytical column was a Supelcosil reverse-phase C18, 15 cm×0.46 cm ID, 3 μm (Supelco, Inc., Bellefonte, Pa.). Mobile phase, consisting of filtered 2 g/L lithium perclorate in methanol/ethanol mixture (40:60, v:v), was used at a flow rate of 1 mL/minute.

Determination of PC

Phosphatidylcholine was essentially determined according to Frei et al. An analytical column was a Supelcosil LCSi, 25 cm×0.46 cm ID, 5 μm (Supelco). A Jasco HPLC System (Jasco Corporation, Tokyo, Japan) provided with two PU-980 pumps, and a UV detector set at 205 nm wavelength was used for detention of total PC.

Statistical Analysis

Statistical analysis was performed using the SAS Statistical Package (SAS Institute Inc., Cary, N.C.). Results are reported as means ±SD. Differences among the samples were evaluated by Student's t-test, and the Kolmogorov-Smirnov and Shapiro-Wilks tests were used to appraise whether the data were random samples from a normal distribution. Finally, the Cramer's index of association was used to evaluate the degree of association among the variables.

CoQ 10 And PC Determinations

CoQ10 levels increased in seminal plasma after treatment, the mean value rising significantly from 42.0±5.1 ng/mL at baseline to 127.1±1.9 ng/mL after 6 months of exogenous CoQ10 administration (P<0.005). A significant increase of CoQ10 content was also detected in sperm cells (from 3.1±0.4 to 6.5±0.3 ng/106 cells, P<0.05). Similarly, PC levels increased significantly both in seminal plasma and sperm cells after treatment (from 1.49±0.50 to 5.84±1.15 μM, P<0.05; and from 6.83±0.98 to 9.67±1.23 nmol/106 cells, P<0.05, respectively) (Table 1).

TABLE 1
Coenzyme Q10 (CoQ10) and phosphatidylcholine (PC) levels in
seminal plasma and sperm cells, baseline and after treatment.

| | Baseline | After Treatment |
|---|---|---|
| CoQ10, seminal plasma (ng/mL) | 42.0 ± 5.1 | 127.1 ± 1.9^{a} |
| CoQ10, sperm cells (ng/10⁶ cells) | 3.1 ± 0.4 | 6.5 ± 0.3^{b} |
| PC, seminal plasma (μM) | 1.49 ± 0.50 | 5.84 ± 1.15^{b} |
| PC, sperm cells (nmol/10⁶ cells) | 6.83 ± 0.98 | 9.67 ± 1.23^{b} |

^{a}After treatment vs. baseline; P < 0.005.
^{b}After treatment vs. baseline; P < 0.05.

Sperm Output

With regard to semen features, a significant difference was found in the forward (class a+b) motility of sperm cells after 6 months of CoQ10 dietary implementation (from 9.13%±2.50% to 16.34%±3.43%, P<0.05) (Table 2). The improvement of motility was also confirmed after the computer-assisted determination of kinetic parameters. A significant increase of VCL (from 26.31±1.50 to 46.43±2.28 μm/second, P<0.05) and VSL (from 15.20±1.30 to 20.40±2.17 μm/second, P<0.05) was found after treatment. No significant differences were found in sperm cell concentration and morphology (Table 2).

TABLE 2
Semen parameters (concentration, motility, and morphology), including
compouter-assisted sperm analysis kinetic features baseline, after 6 months of coenzyme
Q10 dietary implementation and 6 months of wash-out.

| | Baseline | After Treatment | After Wash-out |
|---|---|---|---|
| Sperm concentration (106/mL) | 27.60 ± 7.41 | 25.92 ± 5.63, NS | 26.18 ± 4.04, NS |
| Forward Motility (%) | 9.13 ± 2.50 | 16.34 ± 3.43^{a} | 9.50 ± 2.28^{b} |
| Teratozoospermia (%) | 69.76 ± 4.63 | 67.82 ± 7.44, NS | 66.23 ± 5.34, NS |
| Curvilinear velocity (um/second) | 26.31 ± 1.50 | 46.43 ± 2.28^{a} | — |
| Straight progressive velocity (um/second) | 15.20 ± 1.30 | 20.40 ± 2.17^{a} | — |

Note:
NS = not significant.
^{a}After treatment vs. baseline; P < 0.05.
^{b}After treatment vs. baseline; P < 0.0001.

Interestingly, although a direct correlation was not found (data not shown), a positive dependence (using the Cramer's index of association) was evident among the relative variations, baseline and after treatment, of seminal plasma or intracellular CoQ10 content and of CASA (VCL and VSL) kinetic parameters (Cramer's V=0.4637, 0.3818, 0.3467, and 0.5148, respectively) (Table 3).

TABLE 3
Two-way contingency tables. Percent variation after and
before treatment between VCL (μm/sec) and cellular CoQ10.

| VCL CoQ₁₀ | (−10%; 0%) | (0%; 10%) | (10%; 20%) | (20%; 30%) | (30%; +) | Total |
|---|---|---|---|---|---|---|
| (50%; 100%) | 1 | 1 | 3 | 2 | 1 | 8 |
| (100%; 150%) | 1 | 0 | 3 | 3 | 2 | 9 |
| (150%; 200%) | 0 | 0 | 0 | 2 | 0 | 2 |
| (200%; +) | 0 | 2 | 1 | 0 | 0 | 3 |
| Total | 2 | 3 | 7 | 7 | 3 | 22 |
| Cramer's V = 0.4637 | | | | | | |

Percent variation after and before treatment between VSL (μm/sec) and cellular CoQ₁₀

| VSL CoQ₁₀ | (0%; 10%) | (10%; 0%) | (20%; 30%) | (30%; 40%) | (40%; 50%) | (50%; +) | Total |
|---|---|---|---|---|---|---|---|
| (50%; 100%) | 0 | 1 | 1 | 2 | 2 | 2 | 8 |
| (100%; 150%) | 1 | 1 | 2 | 2 | 2 | 1 | 9 |
| (150%; 200%) | 0 | 0 | 0 | 0 | 0 | 2 | 2 |
| (200%; +) | 0 | 0 | 1 | 1 | 0 | 1 | 3 |
| Total | 1 | 2 | 4 | 5 | 4 | 6 | 22 |
| Cramer's V = 0.3818 | | | | | | | |

Percent variation after and before treatment between VCL (μm/sec) and pharmatic CoQ₁₀

| VCL CoQ₁₀ | (−10%; 0%) | (0%; 10%) | (10%; 20%) | (20%; 30%) | (30%; +) | Total |
|---|---|---|---|---|---|---|
| (0%; 100%) | 0 | 1 | 2 | 1 | 0 | 4 |
| (100%; 200%) | 1 | 0 | 2 | 1 | 0 | 4 |
| (200%; 300%) | 1 | 2 | 1 | 3 | 2 | 9 |
| (300%; 400%) | 0 | 0 | 1 | 0 | 0 | 1 |
| (400%; +) | 0 | 0 | 1 | 2 | 1 | 4 |
| Total | 2 | 3 | 7 | 7 | 3 | 22 |
| Cramer's V = 0.3467 | | | | | | |

Percent variation after and before treatment between VSL (μm/sec) and pharmatic CoQ₁₀

| VSL CoQ₁₀ | (0%; 10%) | (10%; 20%) | (20%; 30%) | (30%; 40%) | (40%; 50%) | (50%; +) | Total |
|---|---|---|---|---|---|---|---|
| (0%; 100%) | 0 | 0 | 0 | 2 | 1 | 1 | 4 |
| (100%; 200%) | 1 | 0 | 0 | 1 | 2 | 0 | 4 |
| (200%; 300%) | 0 | 2 | 2 | 1 | 0 | 4 | 9 |
| (300%; 400%) | 0 | 0 | 0 | 1 | 0 | 0 | 1 |
| (400%; +) | 0 | 0 | 2 | 0 | 1 | 1 | 4 |
| Total | 1 | 2 | 4 | 5 | 4 | 6 | 22 |
| Cramer's V = 0.5148 | | | | | | | |

Note:
CoQ₁₀ = coenzyme Q₁₀;
VCL = curviliness velocity;
VSL = straight progressive velocity.

Sperm forward motility was significantly reduced after 6 months of wash-out (from 16.34% ±3.43% to 9.50% ±2.28%, P<0.001), while no significant differences were found in sperm cell concentration and morphology (Table 2).

In an attempt to verify whether different responses were evident as functions of age, the relative variations (before and after treatment) of CoQ10 and PC content in seminal plasma and sperm cells, as well as forward motility, were analyzed, but no dependence was found (data not shown).

Spontaneous Pregnancy

Three out of 22 patients (13.6%) achieved spontaneous pregnancy within 3 months of the discontinuation of therapy (a 2.4% pregnancy rate per cycle).

Safety Assessment

CoQ10 oral administration was generally well tolerated, and no laboratory abnormalities were observed.

Reduced levels of CoQ10 are found in the seminal plasma and sperm cells of infertile men with idiopathic and varicocele-associated asthenospermia. These results indicate that CoQ10 was one of the compounds contributing to the total antioxidant buffer capacity of semen, and its reduction impaired the ability of the system to deal with oxidative stress.

The data of the present study show a significant improvement of sperm cell kinetic features after 6 months of administration of CoQ10, on the basis of both manual and computer-assisted evaluation. Furthermore, these results are the first to demonstrate that exogenous administration of CoQ10 leads to increased levels in seminal plasma and in sperm cells.

The increment was relevant, especially in seminal plasma where post-treatment levels were 3 times higher than basal ones. Similar increases of CoQ10 concentration (2-3 times higher compared with baseline value) are commonly found in blood plasma after chronic administration of the quinone. Because CoQ10 is a highly lipophylic molecule, it can be reasonably hypothesized that it can diffuse through the phospholipid bilayer of cellular membranes, but presently it is not known whether transport from blood plasma to testicular and accessory male genital glands is a passive one or one that involves an active mechanism.

While not being limited to any particular theory, the Applicants hypothesize that the good degree of association according to the Cramer's V index of association, supports the hypothesis of a pathogenetic role of CoQ10 in asthenospermia based on previously reported data. The improvement in spontaneous pregnancy rates also suggests a benefit of this therapeutic approach.

While not being limited to any particular theory, the positive effect of exogenous administration could be explained on the basis of the well-known involvement of CoQ10 in mitochondrial bioenergetics and of its widely recognized antioxidant properties. Regarding the first point, it is well-known that the mitochondrial concentration of CoQ10 in mammals is close to its K_{M}, as far as NADH oxidation is concerned, and therefore is not kinetically saturating. In these conditions, a small increase in mitocondrial CoQ10 could lead to a relevant rise in respiratory velocity. The resulting improvement of oxidative phosphorylation might well affect sperm cells. Since low PC levels in semen were found to be related to a reduction of the phospholipid pool and to low antioxidant capacity, the increased PC content in semen after treatment might reasonably involve the restoration of scavenger equilibrium. Another possible reason for this finding is that increased levels of CoQ10 also need an appropriate, high concentration of a lipid carrier.

In conclusion, the administration of CoQ10 may play a positive role in treatment of asthenospermia, probably related both to its role in the mitochondrial respiratory chain and to its antioxidant role. The increased concentration of CoQ10 in seminal plasma and sperm cells, the improvement of semen kinetic features after treatment, and the evidence of a direct correlation between CoQ10 concentrations and sperm motility strongly support a cause/effect relationship.

What is claimed is:

1. A method for improving sperm health in a male individual comprising:
administering an effective amount of a composition comprising ubiquinone, ubiquinol or a combination thereof to a male human in need thereof.

2. The method of claim 1, wherein said male suffers from a condition selected from the group consisting of idiopathic asthenospermia, varicocele asthenospermia, teratospermia, prostato-vesiculo-epididymitis, decreased sperm motility, reduced sperm oxidative capacity, reduced seminal plasma or seminal fluid CoQ10 levels, reduced sperm CoQ10 levels, reduced sperm phosphatidylcholine (PC) levels, reduced seminal plasma or seminal fluid PC levels, reduced sperm concentration, and combinations thereof.

3. The method of claim 1, wherein the composition comprises from about 40 mg/day to about 4 g/day of ubiquinone, ubiquinol, or a combination of both.

4. The method of claim 3, wherein the composition comprises from about 100 mg/day to about 1600 mg/day of ubiquinone, ubiquinol, or a combination of both.

5. The method of claim 4, wherein the composition comprises from about 200 mg/day to about 800 mg/day of unbiquione, ubiquinol, or a combination of both.

6. The method of claim 5, wherein said composition also comprises a member chosen from the group consisting of a reducing agent, a solubilizer, an emulsifier, a surfactant, a triglyceride, an oil, a fat, a lipid, a phospholipid, a carbohydrate, an antioxidant, lipoic acid, a carnitine, creatine, an herbal extract, a vitamin, a coenzyme, a mineral, an electrolyte, another biologically active agent, water, a carrier, an excipient, and any combination thereof.

7. A method for treating or preventing male infertility in an individual comprising:
administering an effective amount of a composition comprising ubiquinone, ubiquinol or a combination thereof to a male suffering from a condition selected from the group consisting of idiopathic asthenospermia, varicocele asthenospermia, teratospermia, prostato-vesiculo-epididymitis, decreased sperm motility, reduced sperm oxidative capacity, reduced seminal plasma or seminal fluid CoQ10 levels, reduced sperm CoQ10 levels, reduced sperm phosphatidylcholine levels, reduced sperm concentration, and combinations thereof.

8. The method of claim 7, wherein the composition comprises from about 40 mg/day to about 4 g/day of ubiquinone, ubiquinol, or a combination of both.

9. The method of claim 8, wherein the composition comprises from about 100 mg/day to about 1600 mg/day of ubiquinone, ubiquinol, or a combination of both.

10. The method of claim 9, wherein the composition comprises from about 200 mg/day to about 800 mg/day of unbiquione, ubiquinol, or a combination of both.

11. The method of claim 10, wherein said composition also comprises a member chosen from the group consisting of a reducing agent, a solubilizer, an emulsifier, a surfactant, a triglyceride, an oil, a fat, a lipid, a phospholipid, a carbohydrate, an antioxidant, lipoic acid, a carnitine, creatine, an herbal extract, a vitamin, a coenzyme, a mineral, an electrolyte, another biologically active agent, water, a carrier, an excipient, and any combination thereof.

12. A method for improving the oxidative capacity of sperm comprising:
administering an effective amount of a composition comprising ubiquinone, ubiquinol or a combination thereof to a male human in need thereof.

13. The method of claim 12, wherein the composition comprises from about 40 mg/day to about 4 g/day of ubiquinone, ubiquinol, or a combination of both.

14. The method of claim 13, wherein the composition comprises from about 100 mg/day to about 1600 mg/day of ubiquinone, ubiquinol, or a combination of both.

15. The method of claim 14, wherein the composition comprises from about 200 mg/day to about 800 mg/day of unbiquione, ubiquinol, or a combination of both.

16. The method of claim 15, wherein said composition also comprises a member chosen from the group consisting of a reducing agent, a solubilizer, an emulsifier, a surfactant, a triglyceride, an oil, a fat, a lipid, a phospholipid, a carbohydrate, an antioxidant, lipoic acid, a carnitine, creatine, an herbal extract, a vitamin, a coenzyme, a mineral, an electrolyte, another biologically active agent, water, a carrier, an excipient, and any combination thereof.
